(19) United States
(12) Patent Application Publication
(10) Pub. No.: US 2011/0263018 A1
(43) Pub. Date: Oct. 27, 2011

(54) CORE-SHELL STRUCTURED DELIVERY SYSTEM FOR GROWTH FACTORS, A PREPARATION METHOD THEREOF, AND USE THEREOF FOR THE DIFFERENTIATION OR PROLIFERATION OF CELLS

(75) Inventors: Kwideok PARK, Seoul (KR); Dong Keun Han, Seoul (KR); Dong Hoon Choi, Daejeon (KR); Jae-Jin Kim, Seoul (KR); Hee Joong Kim, Seoul (KR); Heung Jae Chun, Seoul (KR)

(73) Assignee: KOREA INSTITUTE OF SCIENCE AND TECHNOLOGY, Seongbuk-gu (KR)

(21) Appl. No.: 12/902,537

(22) Filed: Oct. 12, 2010

(30) Foreign Application Priority Data
Apr. 26, 2010 (KR) 10-2010-0038221

(51) Int. Cl. C12N 5/02; C12N 5/00

(52) U.S. Cl. 435/377; 435/375

(57) ABSTRACT

The present invention relates to a method of preparing a delivery system capable of loading bioactive growth factors that are essential for the differentiation and proliferation of cells and is characterized as loading at least two types of components comprising growth factors in a single carrier, whereby the release of each of the plurality of growth factors can be temporally controlled. Specifically, the method of preparing a microcapsule type growth factor delivery system according to the present invention includes: (1) preparing a polymeric microsphere comprising a first component, and then encapsulating the microspheres by electrodropping the polymer microsphere into another polymer comprising a second component, thereby manufacturing a core-shell structured, microcapsule type delivery system, or (2) encapsulating a polymer solution comprising a first component by electrodropping the polymer solution into another polymer comprising a second component, thereby manufacturing a core-shell structured microcapsule type delivery system. The present invention also provides a stem cell differentiation method involving bringing a microcapsule type delivery system loaded with multiple growth factors according to the present invention into contact with stem cells.

The present application claims priority from Korean Patent Application No. 10-2010-0038221, filed on Apr. 26, 2010, the subject matter of which is incorporated herein by reference in its entirety.

### FIELD OF THE INVENTION

The present invention relates to a delivery system in a microcapsule form which is capable of effectively delivering active biomolecules including growth factors that are essential for differentiation and proliferation of cells, as well as a method of manufacturing the same. The present invention is expected to greatly enhance the proliferation and differentiation efficiencies of stem cells and tissue cells.

### BACKGROUND OF THE INVENTION

Currently, basic and applied research relating to the induction and control of stem cell differentiation, a core field of regenerative medicine, is being conducted extensively, and clinical studies of stem cells have also been actively underway.

However, to date, there is still little understanding regarding the external growth factors affecting the differentiation of stem cells. For example, there is almost no fundamental understanding or information regarding the concentrations, time periods, and application positions necessary for the in vitro or in vivo application of growth factors that are required for stem cell differentiation.

Accordingly, it is common to design growth factor-loaded delivery systems, if possible, such that the drug can be control-released over a long period of time. However, delivery systems with such a design have to carry large amounts of growth factors which may be harmful to cells. In addition, the continuous exposure to growth factors may lead to significantly reduced sensitivity of stem cells to external signals and may further induce undesirable differentiation.

Therefore, the key in a growth factor delivery system is maximizing the differentiation efficiency of stem cells with a minimum amount, where it is necessary that the growth factor delivery is different depending on the time and position. That is because, rather than being exposed to growth factors all the time, stem cells in the human body are supposed to possess an extremely economic mechanism in accomplishing a given objective with a minimum exposure at appropriate time and position. Accordingly, in order to mimic such in vivo mechanism of stem cells, a delivery system capable of carrying out temporally different delivery or localized delivery of multiple growth factors, not a single growth factor, to stem cells is required.

According to conventional technology, growth factors were released from a delivery system comprising micro- or nano-sized microspheres where growth factors were attached onto the surface or loaded inside, or by physical mixing between polymers and growth factors. Synthetic or natural polymers approved for biocompatibility have been used for manufacturing delivery systems. The delivery system was manufactured in a single or composite form.

The most common form is manufacturing microspheres using a polymer. As for microsphere fabricating methods using biodegradable polymers, phase-separation methods (U.S. Pat. No. 4,675,189), spray-drying methods (U.S. Pat. No. 6,709,650), solvent-evaporation-drying methods (U.S. Pat. No. 4,652,441), and low temperature solvent extraction methods (U.S. Pat. No. 5,019,400) were published. In addition, there are methods using water-soluble organic solvents such as acetic acid, lactic acid, acetone, etc., instead of dichloromethane or chloroform as the polymer solvent, whereby bio- and cytocompatibility are improved, and drug capacity is further enhanced (U.S. Pat. No. 5,100,699).

Meanwhile, research articles have been published on inducing the differentiation of stem cells using a scaffold in which a microsphere comprising growth factors is incorporated, or using a single microsphere. Chen et al. manufactured a scaffold by encapsulating two growth factors i.e., bone morphogenic protein-2 (BMP-2) and insulin-like growth factor (IGF), in gelatin microspheres, mixing the BMP-2/IGF gelatin microspheres with a hydrogel, and subjecting the mixture to freeze-drying (Chen et al., Biomaterials, 2009). Elisseff et al. incorporated a transforming growth factor-beta (TGF-β) and IGF into lactic acid-glycolic acid copolymer (PLGA) microspheres, and then introduced the microspheres in a hydrogel to culture a cartilage cell, and investigated the effect (Elisseff et al., J Orthopedic Research, 2001). Meanwhile, Jakelene et al. reported distinctive release patterns for the respective growth factors by manufacturing PLGA microspheres comprising each growth factor and forming a scaffold comprising these microspheres in a three-dimensional agglomerate (Jakelene et al., Biomaterials, 2008). In addition, Park et al. confirmed the differentiation capability of stem cells by preparing gelatin microparticles comprising a transforming growth factor and fabricating a composite comprising the particles with a hydrogel (Park et al., Biomaterials, 2007).

However, most of the above-mentioned approaches are directed to a delivery system comprising a single growth factor, or even if two kinds of growth factors were used, to a system where the growth factors were loaded in a single particle, in which case there is a limitation in delivering the two kinds of growth factors to stem cells by temporally distinguishing their release patterns, although the controlled release of the two growth factors may be possible. The distinguishing feature of the present invention over prior art lies in the fact that temporal release of multiple growth factors from a single microcapsule is possible by respectively loading the multiple growth factors into core- shell. There have been few reports on the effect of such temporal release of growth factors on stem cells. Accordingly, the microcapsule growth factor delivery system according to the present invention is expected to be very helpful in studying the mechanism of stem cell differentiation on a temporal basis.

### SUMMARY OF THE INVENTION

It is, therefore, an objective of the present invention to develop a microcapsule-type delivery system capable of temporal release of multiple components which are necessary for cell differentiation or proliferation, including growth factors, and thereby overcome the problems of conventional growth factor delivery systems and enhance the efficacy of stem cell differentiation into a specific lineage.

### Technical Means to Solve Problems

The present invention relates to a delivery system capable of loading two kinds of components which are necessary for differentiation or proliferation of cells, including growth factors, and providing a distinctive temporally distinguishable release for each component. Specifically, the present invention provides a microcapsule-type system for growth factor delivery which has a core-shell structure in which the two components are loaded in the core and shell, respectively. Compared to conventional growth factor delivery systems, the present invention not only provides the temporal release of multiple growth factors from a single delivery system, thereby more effectively inducing or controlling stem cell differentiation, but is also helpful in understanding the mechanisms of stem cell differentiation. Further, a growth factor delivery system according to the present invention can be effectively applied to the proliferation of tissue cells, thus contributing to the promotion of the regeneration using tissue cells.

The term “microcapsule” used herein refers to a particle in which solid or pharmaceutically active materials are located in the core. The microcapsule according to the present invention has a diameter ranging from approximately 250 to 450 um, specifically from 100 to 200 um. If the diameter of the microcapsules is too large, there may be limitations in the various applications, for example, making a composite 3D scaffold for tissue implantation. If the diameter is too small, the microcapsules are not easy to handle and controlling the release of the growth factors may be difficult.

The above core and shell may be made of any biodegradable polymers as long as they are not toxic to human beings, and may specifically be biogradable synthetic or natural polymers.

Representative synthetic polymers that can be used for the present invention may include, but are not limited to, poly(glycolic acid) (PGA), poly(L-lactic acid) (PLLA), poly(lactic acid-co-glycolic acid) (PLGA), poly-ε-caprolactone (PCL), poly(L-lactic acid-co-caprolactone) (PLCL), poly(amino acid), polyanhydride, polyorthoester, polyethylene glycol, polyvinyl alcohol, biogradable polyurethane, and copolymers thereof. The above biodegradable polymers have a molecular weight ranging from 5,000 to 1,000,000 g/mol, more specifically 10,000 to 500,000 g/mol, but are not limited thereto.

Representative natural polymers that can be used for the present invention may include, but are not limited to, collagen, alginate, gelatin, chitosan, fibrin, hyaluronic acid (HA), hyaluronic acid derivatives, cellulose, cellulose derivatives, self-assembled peptides, and composites thereof.

The present invention relates to a core-shell structured, microcapsule type delivery system in which two components that are necessary for differentiation or proliferation of cells, including growth factors, are loaded in the core and shell, respectively. The growth factors may be at least one selected from, for example, transforming growth factor-beta (TGF-β), fibroblast growth factor (FGF), bone morphogenic protein (BMP), vascular endothelial growth factor (VEGF), epidermal growth factor (EGF), insulin-like growth factor (IGF), platelet-derived growth factor (PDGF), nerve growth factor (NGF), hepatocyte growth factor (HGF), epidermal growth factor, angiopoietin-1, angiopoietin-2, neurotrophins, placental growth factor (PIGF), granulocyte colony stimulating factor (G-CSF), granulocyte macrophage colony stimulating factor (GM-CSF), but are not limited thereto.

The two components loaded in the growth factor delivery system according to the present invention may both be growth factors, or only one may be a growth factor. When both of the two components are growth factors, it is desirable that they are different from each other.

When one of the two components loaded in the growth factor delivery system according to the present invention is a growth factor, the other component may be at least one selected from, for example heparin, animal growth hormone, human growth hormone, erythropoietin (EPO), interferon, follicle-simulating hormone (FSH), luteinizing hormone, goserelin acetate, leuprolein acetate, luteinizing hormone-releasing hormone (LH-RH) agonist of decapeptyl, dexamethasone, ascorbate-2-phosphate, β-glycerophosphate, insulin, glucose, paclitaxel, rapamycin, anti-inflammatory agents, and the like, but are not limited thereto. However, the “multiple growth factors” mentioned in the present application should be interpreted to include the above components.

Stem cells mentioned herein refer to cells that remain undifferentiated while retaining the capability to differentiate into all types of cells comprising the body, such as blood vessels, neurons, myocardium, blood, cartilage, bone etc. Examples of such stem cells may include embryonic stem cells, bone marrow stem cells, adipose stem cells, umbilical cord blood stem cells, peripheral blood stem cells, hematopoietic stem cells, muscle stem cells, neural stem cells, induced pluripotent stem cells, etc. As such, stem cells can differentiate only when appropriate growth factors are delivered at the desired site of action. In the present invention, two components that are necessary for differentiation or proliferation of cells, including growth factors, are loaded in a core and a shell, respectively, thereby delivering multiple growth factors to stem cells at different times or at different sites. Accordingly, a delivery system capable of effectively mimicking a mechanism of in vivo differentiation of stem cells can be provided.

If a microcapsule-type delivery system in which multiple growth factors are respectively loaded in a core and a shell according to the present invention is used, it may be possible to maximize the differentiation efficiency of stem cells with even a small amount of growth factors. Further, cytotoxicity can be minimized since large amounts of growth factors are not loaded, while lowered sensitivity of stem cells to external signals that is caused by their exposure to high amounts of growth factors can be prevented. Further, the possibility of stem cells being induced to undesirable differentiation due to large amounts of growth factors can be reduced.

In order to further delay the release of the growth factor loaded in a shell, a microcapsule-type growth factor delivery system can be coated. The coating layer for the shell portion can be formed by inducing physical adsorption or chemical reaction, while the thickness thereof can be manipulated. Once the coating layer is formed, the release rate resulting from growth factor diffusion within the microcapsule can be greatly reduced. Such coating may include, for example, chitosan, protamine, gelatin, collagen, polyethylene imine (PEI), poly-L-lysine, dextran sulfate, hyaluronic acid, etc., but is not limited thereto.

In order to enhance the mechanical properties of the microcapsule-type growth factor delivery system according to the present invention, microcapsules that have been prepared can be treated with a crosslinking agent. The above crosslinking agent may be, for example, ethyldimethylaminopropyl carbodiimide, genipin, glutaraldehyde, but is not limited thereto. However, for biocompatibility, it is desirable to exclude the highly toxic ones.

The present invention also relates to a method of preparing a core-shell structured, microcapsule type growth factor delivery system which can load two components that are necessary for differentiation or proliferation of cells, including growth factors. Specifically, the method of preparing a microcapsule type growth factor delivery system according to the present invention includes: (1) preparing a polymer microsphere comprising the first component that is necessary for differentiation or proliferation of cells, and then making a polymer microcapsule by electrodropping the polymer microsphere to another polymer comprising the second component that is necessary for differentiation or proliferation of cells, thereby manufacturing a core-shell structured, microcapsule type delivery system, or (2) encapsulating a polymer solution comprising the first component that is necessary for differentiation or proliferation of cells by electrodropping the polymer solution into another polymer comprising the second component that is necessary for differentiation or proliferation of cells, thereby manufacturing a microcapsule type delivery system.

The first and second components may both be growth factors, or only one may be a growth factor. With respect to these components, the above described explanations may be referred to.

In accordance with one embodiment of the present invention, i.e., a method of preparing a microcapsule type growth factor delivery system, a polymer microsphere carrying the first component that is necessary for differentiation or proliferation of cells is prepared and then the polymer microsphere is encapsulated by electrodropping it into another polymer layer comprising the second component that is necessary for differentiation or proliferation of cells, thereby manufacturing a core-shell structured microcapsule type delivery system.

The microsphere can be prepared by using known methods, such as phase-separation method, spray-drying method, solvent evaporation-drying method, and low temperature solvent extraction method. The “microsphere” mentioned herein refers to a solidified particle that is obtained by emulsifying (dispersing) a polymer dissolved in a solvent with a bioactive material to form an emulsion, and then curing the polymer by solvent vaporization.

In a specific embodiment, the microsphere can have a structure with multiple pores interconnected inside and a covered surface, but is not limited thereto. The surface-covered structure allows the control of the excessive release of the first component loaded in the core portion of the microcapsule type growth factor delivery system in the early stages of administration, while the porous structure inside the microsphere makes it possible to gradually release the first component for a prolonged period. When the present invention is practiced according to the above embodiment, it is desirable that the microsphere has a size ranging from 50 to 100 um and a porosity ranging from at least 0 to at most 98%. The preparation method for the microsphere as described above is described in detail in Korean Patent Publication No. 10-2009-0131975, the subject matter of which is incorporated herein by reference in its entirety, and U.S. Patent Application Publication No. 2009/0317478.

In a microcapsule-type growth factor delivery system according to the present invention, if the core is made of a polymer microsphere, various shapes of microspheres can be premade and loaded into the microcapsule. In addition, if the release pattern varies depending on the shape of the microsphere, the shape of the microsphere can be optimized, and thus a microcapsule providing desired release profiles with credibility can be prepared.

In accordance with another embodiment of a method of preparing a microcapsule type growth factor delivery system according to the present invention, a polymer solution carrying the first component that is necessary for differentiation or proliferation of cells is encapsulated by electrodropping it into another polymer carrying the second component that is necessary for differentiation or proliferation of cells, thereby manufacturing a core-shell structured, microcapsule type delivery system.

The polymer solution carrying the first component can be prepared by simultaneously adding a biogradable polymer and the first component in an organic solvent, or dissolving only the biodegradable polymer into an organic solvent and then suspending the first component thereto.

Suitable organic solvents that can be used for dissolving the biogradable polymer, which may vary depending on the polymer type, may include, for example, methylenechloride, chloroform, carbon tetrachloride, acetone, dioxane, tetrahydrofuran, hexafluoroisopropanol, etc. Of the organic solvents exemplified above, it is more desirable to use methylenechloride and chloroform.

In a microcapsule type growth factor delivery system according to the present invention, if the core is made using a polymer solution, it is possible to accurately construct a single core and a shell encapsulating the core. In addition, controlling the entire size of the microcapsule is easier.

In the present invention, it is desirable to encapsulate the polymer microsphere or polymer solution carrying the first component into the polymer carrying the second component by electrodropping, but it is not limited thereto.

Electrodropping is a method of dispersing a liquid into droplets using electric force and is used in the preparation of micro- and nano-sized particles. Specifically, a polymer solution is electrically charged by applying a high voltage electric field and the charged polymer solution is sprayed through a microneedle. The polymer solution migrating through the microneedles forms a cone-jet at the tip of the needle, where polymers having high viscosity are dispersed into a fiber form, while polymers with appropriate viscosity are dispersed into micro-sized spherical particles.

The conditions for electrodropping may change depending on the type, molecular weight, and flow rate of the subject polymer, applied voltage, etc. In the present invention, the voltage ranges from 5,000 to 10,000 V and the spray rates range from 0.1 to 1.2 ml/h and from 0.03 to 0.5 ml/h in the core and shell, respectively. The nozzle sizes of the core and the shell are from 20 to 26 gauges and from 15 to 18 gauges, respectively. In a coaxial spraying system, the nozzle size of the core must be smaller than that of the shell.

The above electrodropping may employ a coaxial spraying system. Specifically, a polymer microsphere or polymer solution loading the first component is dropped through an inner needle, and a polymer solution carrying a second component flows through an outer needle. Starting from the outer surface, the polymer solution carrying the second component is crosslinked to form a shell structure.

When the core is made using a polymer microsphere, as the shell structure is formed as described above, the microcapsule-type delivery system having a core-shell structure in which the first component is loaded inside and the second component is loaded outside, is prepared. However, if the core is made using a polymer solution, although the shell structure is formed as above, the inner side exists in a liquid state still with an organic solvent. Thus, in order to remove the organic solvent and induce solidification of the polymer, additional treatments such as solvent vaporization may be necessary.

The polymer microsphere or polymer solution carrying the first component, and the polymer solution carrying the second component are released at the same time, which is essential for the formation of a core-shell structure in a microcapsule. Polymer solutions flow through different needles, but are combined at the coaxial needle tip right before being released and dropped while forming a core-shell structure.

The present invention also provides a method of identifying the distinctive release behaviors of multiple growth factors, including using a microcapsule-type delivery system manufactured according to the methods exemplified above in order to identify in vitro distinctive release behaviors for multiple growth factors. The above method can include changing the places where the growth factors are loaded in the core and the shell and comparing the release patterns after and before the change. This method not only allows the identification of the individual release behaviors of the multiple growth factors, but also enables the selection of a biodegradable polymer capable of optimally controlling the release behavior of a specific growth factor combination.

The present invention also provides a method of determining the capability of a plurality of growth factors in stem cell differentiation, including using a microcapsule-type delivery system manufactured according to the methods exemplified above and identifying the effect of a plurality of growth factors on the differentiation of stem cells in vitro. The effects of temporal delivery of a plurality of growth factors on stem cell differentiation have not been reported yet. Accordingly, with the use of the above method, it is possible to examine the differentiation capability of stem cells resulting from the specific combinations of growth factors more conveniently and easily.

The present invention also provides a method of differentiating stem cells which includes bringing a microcapsule-type delivery system loaded with a plurality of growth factors according to the present invention into contact with stem cells. Due to the use of the above growth factor delivery system according to the present invention, it is possible to effectively differentiate stem cells without using large amounts of expensive growth factors.

As the microcapsule-type delivery system loaded with a plurality of growth factors according to the present invention can be effectively used in the proliferation of tissue cells, it can also contribute to the promotion of the regeneration of damaged tissue.

The microcapsule-type delivery system loaded with a plurality of growth factors according to the present invention maximizes the stem cell differentiation capability and promotes the proliferation and differentiation of tissue cells (e.g., myocardium cells, neural cells, chondrocytes, osteoblasts, osteoclasts, liver cells, pancreatic cells, endothelial cells, epidermal cells, smooth muscle cells, and intervertebral disc cells, etc.). Thus, it may be used in the treatment of incurable diseases and the reconstruction and regeneration of damaged tissues of the human body. Hence, the present invention provides a composition for treatment of incurable diseases and for tissue reconstruction and regeneration, containing as an active ingredient, a microcapsule-type delivery system loaded with a plurality of growth factors according to the present invention.

### Effect of the Invention

The present invention, unlike the conventional delivery systems for a single growth factor, is characterized as a delivery system for multiple growth factors which provides the temporal release of multiple growth factors to stem cells from a single delivery system. By the system according to the present invention which is capable of controlling the release of growth factors in various ways, the differentiation capability of stem cells can be greatly enhanced, and it will also be helpful in studying the mechanisms of stem cell differentiation upon growth factors treatment. In addition, the multiple growth factor delivery system according to the present invention can be used in the treatment of incurable diseases and the reconstruction and regeneration of damaged human tissues, because it maximizes the in vitro differentiation capability of stem cells and leads to the proliferation and differentiation of tissue cells.

### BRIEF DESCRIPTION OF THE DRAWINGS

FIG. 1A is a schematic diagram showing an illustrative coaxial system for preparing a core-shell microcapsule by electrodropping.

FIG. 1B is an optical image of a microcapsule (Example 2) according to the present invention prepared by using the system illustrated in FIG. 1A.

FIG. 1C is a photograph of the coaxial system for encapsulating PLGA emulsion into alginate by electrodropping.

FIG. 2 is an optical image of microcapsules (Example 3) according to the present invention prepared by using the system illustrated in FIG. 1C.

FIG. 3 illustrates the release profiles according to the type of a microcapsule (M1) composed of a PLGA core containing bone morphogenic protein (BMP-2) and an alginate shell containing dexamethasone, and a microcapsule (M2) composed of a PLGA core containing dexamethasone and an alginate shell containing BMP-2.

FIG. 4 illustrates the results from a RT-PCR analysis for investigating the effect of osteogenic differentiation from rat bone marrow derived stem cells, using a microcapsule (M1) composed of a core containing BMP-2 and a shell containing dexamethasone, and a microcapsule (M2) composed of a core containing dexamethasone and a shell containing BMP-2. FIG. 4(A) is a photograph of the mRNA levels of Type I collagen (Col Ia), alkaline phosphatase (ALP), osteocalcin (OC), and osteopontin (OP), and FIG. 4(B) is the gene expression level normalized to GAPDH.

Hereinafter, the present invention will be described in more detail with reference to the following examples. However, it will be apparent to those skilled in the art that the following examples are for illustrative purposes only and that the invention is not intended to be limited by these examples.

### EXAMPLES

### Example 1

### Preparation of Polymer PLGA Microspheres

In the present working example, polymeric microspheres were prepared by a W₁/O/W₂ multiple emulsion method. First, a biodegradable polymer in combination with a biologically active material was suspended in an organic solvent suitable for dissolving a lactic acid-glycolic acid copolymer, a biodegradable PLGA (Boehringer Ingelheim, Germany, 50:50). PLGA (200 mg) was dissolved in 4 ml of chloroform which is an organic solvent, followed by the addition of 4.5% fetal bovine serum (FBS) and 3 mg BMP-2. PVA (4.5%, w/v), a surfactant, was added for emulsification, followed by ultrasonic treatment for 90 seconds. The resulting solution was added to 0.2% PVA solution, and simultaneously the mixture was rapidly stirred at room temperature for 4 hours by means of a stirrer. Finally the organic solvent chloroform was completely evaporated to collect solidified microspheres. The collected microspheres were washed and then subjected to freeze-drying.

### Example 2

### Encapsulation of PLGA Microspheres into Alginate

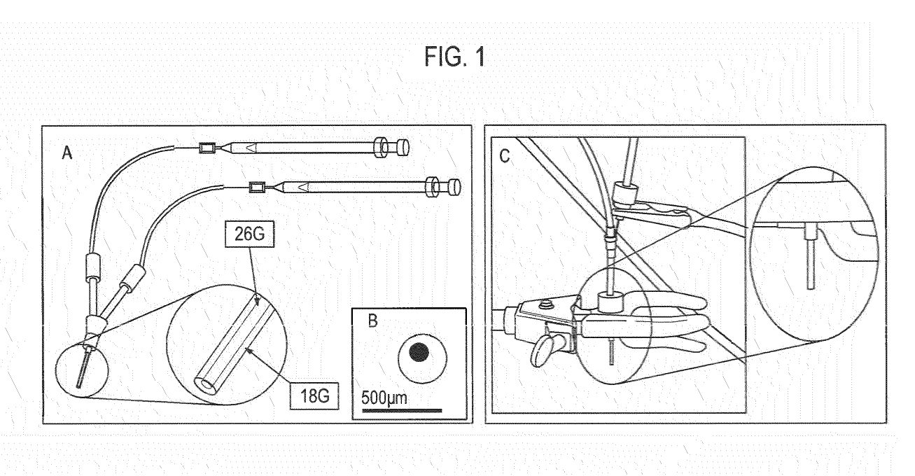

The PLGA microspheres obtained in Example 1 were encapsulated by alginate. The encapsulation of the polymer microspheres using alginate was carried out by electrodropping using a coaxial system. A 20-gauge needle was used as the inner needle of the coaxial system so that a PLGA microsphere of about 100 μm size could pass, while a 17-gauge needle was used outside. The alginate used in the encapsulation had viscosities ranging from 80 to 120 cp and from 500 to 600 cp, respectively. To a 1 mL alginate solution (0.5%, w/v) was added 10 mg of microspheres to form a suspension. The suspension was placed in the inner needle of the coaxial system using a lcc syringe. The 500-600 cp alginate solution in which dexamethasone was added was injected into the outer needle using a 20 cc syringe. The release of the alginate solution from the coaxial system was carried out using two syringe pumps, and the flow rate of each pump was fixed at 0.1 ml/h in the inner needle and at 0.5 ml/h in the outer needle. In order to reduce the size of the microcapsules, a high voltage of 8000V was applied in dropping the alginate solution. The alginate solution was electrodropped to a rotating 1M CaCl₂ solution. Starting from the outside, crosslinking took place where finally a delivery system for multiple growth factors composed of a microcapsule core made of PLGA microspheres comprising BMP-2 and a shell made of an alginate layer comprising dexamethasone was prepared (FIG. 1B). The size of the microcapsules was in the range between 200 and 300 μm.

### Example 3

### Encapsulation of PLGA Polymer Solution into Alginate

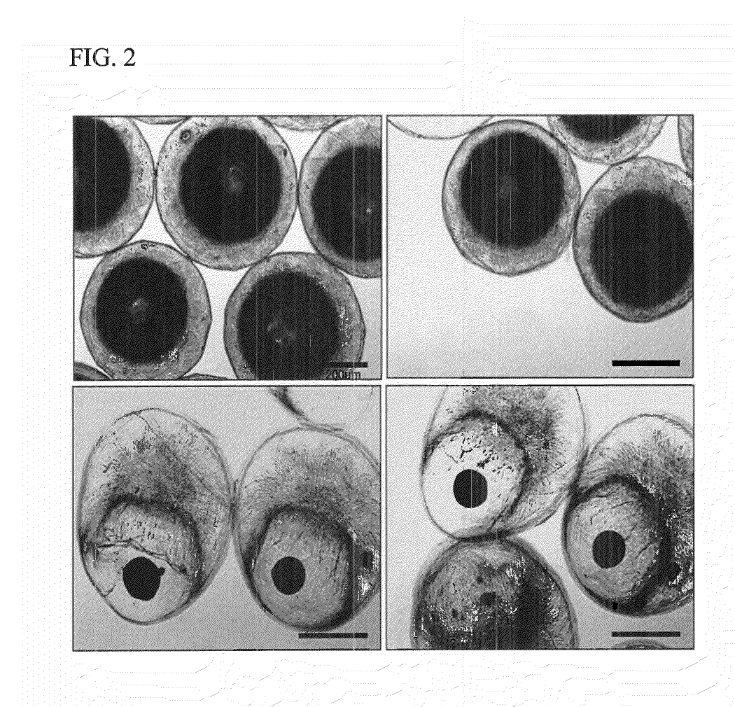

The present example presents a method of manufacturing a delivery system for multiple growth factors comprising a PLGA solution in the core layer instead of PLGA microspheres. PLGA (0.3 g) was dissolved in 8 ml chloroform, followed by the addition of BMP-2 (100 ng), a growth factor. For the homogeneous distribution of growth factors, an ultrasonic homogenizer was operated with 30% output force for 30 seconds to induce an emulsion. The resulting solution was injected to the inner needle of a coaxial system using a 10 cc syringe. In addition, the outer needle was filled with a 0.5% alginate solution (50 ml), supplemented with 5 mg dexamethasone, using a 20 ml syringe. In the same manner as in Example 2, two syringe pumps were used to maintain flow rates in the inner and outer needles at 0.6 ml/h and at 0.3 ml/h, respectively. The alginate solution being escaped from the coaxial system under a high voltage was dropped to a rotating 1M CaCl₂ solution. Starting from the outside, the alginate was crosslinked to form microcapsules comprising a PLGA core and an alginate layer, and the diameter size thereof was approximately 300 to 400 μm. Subsequently, in order to remove the solvent from the PLGA core and induce solidification of the polymer, the microcapsules were stirred in 300 ml PVA (0.25%, w/v) for 4 hours and dried, and then the organic solvent was completely evaporated in a desiccater. Finally, microcapsules composed of a BMP-2-loaded core and a dexamethasone-loaded shell were manufactured (FIG. 2).

### Example 4

### Assay for Release Behavior of Multiple Growth Factors using the Microcapsules According to the Present Invention

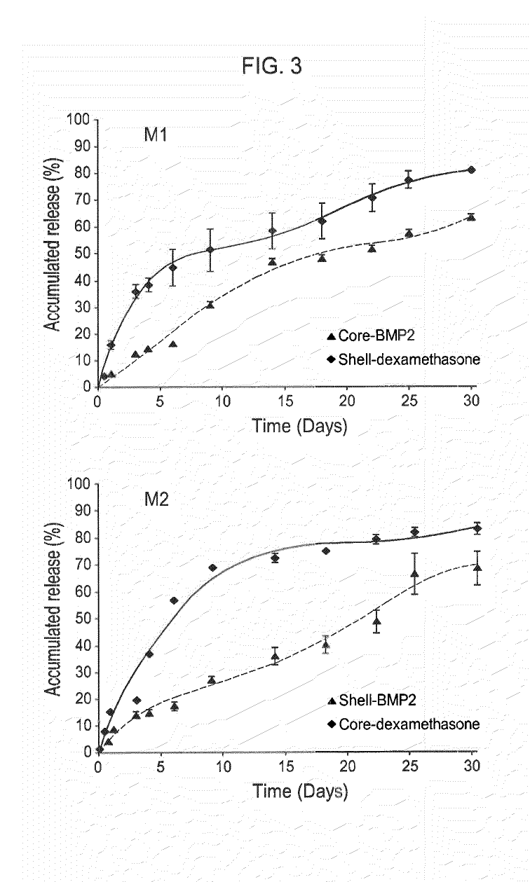

In order to test the release behavior of BMP-2 contained in the core and dexamethasone contained in the shell of a microcapsule-type multiple growth factor delivery system, the microcapsule manufactured in Example 3 was added to a PBS solution and the release pattern was observed for 4 weeks (M1). In addition, the positions of BMP-2 and dexamethasone were exchanged (M2), and the release patterns before and after the change were compared. The results of release pattern are shown in FIG. 3.

### Example 5

### Stem Cell Differentiation using the Microcapsules According to the Present Invention

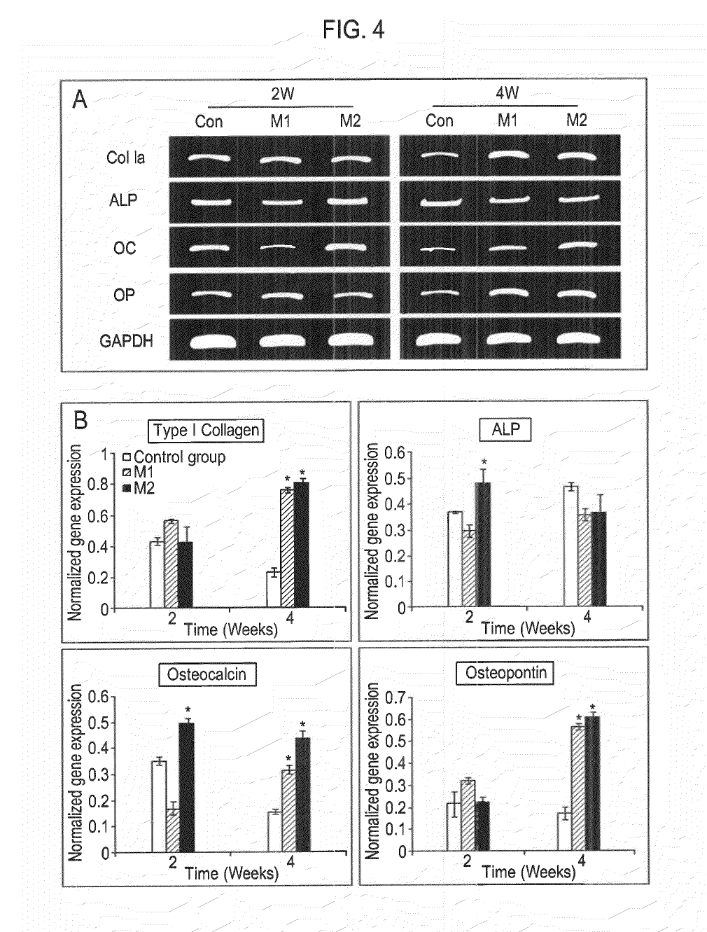

The effect of osteogenic differentiation from rat bone marrow derived stem cells was tested by RT-PCR analysis using a microcapsule (M1) composed of a core containing bone morphogenetic protein (BMP-2) and a shell containing dexamethasone, and a microcapsule (M2) composed of a core containing dexamethasone and a shell containing BMP-2. Specifically, the expression of Type I collagen (Col Ia), alkaline phosphatase (ALP), osteocalcin (OC), and osteopontin (OP), specific markers of osteogenesis, was measured according to time, where the qualitative (FIG. 4A) and quantitative (FIG. 4B) results are illustrated in FIG. 4.

In order to prepare alginate beads with stem cells, 3 ml alginate solution (1%, w/v), rat bone marrow-derived stem cells (BMSC; 5×10⁶), and multiple growth factor-loaded microcapsules (50 mg) were evenly stirred in a clean bench. The alginate mixture was dropped to a rotating 0.1M CaCl₂ solution using a sterilized 5 cc syringe and a 15-gauge needle, whereby the alginate was crosslinked to form a solid bead. For use as a three-dimensional scaffold for the cultivation of stem cells, the alginate beads prepared above were washed with a saline solution and cultivated under different conditions for the individual experimental groups for a certain period. Alginate beads comprising microcapsules that do not carry multiple growth factors were cultivated as a control group in an osteogenic medium, Dulbecco's modified essential medium (DMEM) containing 1% penicillin/streptomycin, 10% fetal bovine serum, 10 mM β-glycerophosphate, 50 μg/ml ascorbate-2-phosphate, dexamethasone for 4 weeks. Meanwhile, as the two experimental groups, alginate beads comprising multiple growth factor-loaded microcapsules (M1 or M2) were cultivated in an incubator at 37[?] for 4 weeks using an osteogenic medium excluding BMP-2 and dexamethasone. Meanwhile, for analysis of the osteogenic gene expression, the beads were added to 1.5 ml tubes, respectively, with the addition of 1 ml of Trizol, and the beads were broken up using a homogenizer. The subsequent process followed the general protocol for isolation of RNA from the cells. The isolated RNA was converted to cDNA by reverse transcription using Maxime RT Premix (Intron) and the synthesized cDNA was used as a template for PCR-analysis of osteocalcin (OC), osteopontin (OP), Type I collagen (Col Ia), alkaline phosphatase (ALP) expression, which are specific markers of osteogenic differentiation.

It is appreciated from the results of gene expression (FIG. 4) that the experimental group comprising multiple growth factor-loaded microcapsules according to the present invention expresses osteocalcin, osteopontin, and Type I collagen relatively strongly compared to the control group. The M1 and M2 experimental groups which exhibit distinctive release patterns were not definitely different in expression, but M2 was found to show relatively strong expression of osteocalcin and ALP at two weeks in particular.

What is claimed:

1. A growth factor delivery system having a core-shell structure in which two components that are necessary for differentiation or proliferation of cells are loaded in the core and the shell, respectively, wherein at least one of said two components is a growth factor.

2. The growth factor delivery system in accordance with claim 1, wherein the two components that are necessary for differentiation or proliferation of cells are all growth factors, wherein each growth factor, independently, is at least one selected from the group consisting of transforming growth factor (TGF-β), fibroblast growth factor (FGF), bone morphogenic protein (BMP), vascular endothelial growth factor (VEGF), epidermal growth factor (EGF), insulin-like growth factor (IGF), platelet-derived growth factor (PDGF), nerve growth factor(NGF), hepatocyte growth factor (HGF), epidermal growth factor, angiopoietin-1, angiopoietin-2, neurotrophin, placental growth factor (PIGF), granulocyte colony simulating factor (G-CSF), and granulocyte macrophage colony simulating factor (GM-CSF).

3. The growth factor delivery system in accordance with claim 1, wherein one of the two components necessary for differentiation or proliferation of cells is a growth factor, and the other component is at least one selected from the group consisting of heparin, animal growth hormone, human growth hormone, erythropoietin, interferon, follicle-simulating hormone, luteinizing hormone, goserelin acetate, leuprolein acetate, luteinizing hormone-releasing hormone agonist of decapeptyl, dexamethasone, ascorbate-2-phosphate, β-glycerophosphate, insulin, glucose, paclitaxel, rapamycin, and an anti-inflammatory agent.

4. The growth factor delivery system in accordance with any one of claims 1-3 which is in a microcapsule form.

5. The growth factor delivery system in accordance with claim 4, wherein the microcapsule has a diameter ranging from 100 to 400 μm.

6. The growth factor delivery system in accordance with any one of claims 1-3, wherein either of the core or shell, or both are prepared from synthetic polymers selected from the group consisting of poly(glycolic acid) (PGA), poly(L-lactic acid) (PLLA), poly(lactic acid-co-glycolic acid) (PLGA), poly-ε-caprolactone (PCL), poly(L-lactic acid-co-caprolactone) (PLCL), poly(amino acid), polyanhydride, polyorthoester, polyethylene glycol, polyvinyl alcohol, biodegradable polyurethane, and copolymers thereof.

7. The growth factor delivery system in accordance with any one of claims 1-3, wherein either of the core or shell, or both are prepared from natural polymers selected from the group consisting of collagen, alginate, gelatin, chitosan, fibrin, hyaluronic acid, hyaluronic acid derivatives, cellulose, cellulose derivatives, self-assembled peptide, and composites thereof.

8. The growth factor delivery system in accordance with any one of claims 1-3, wherein the shell is coated with a material selected from the group consisting of chitosan, protamine, gelatin, collagen, poly(ethyleneimine) (PEI), poly-L-lysine, dextran sulfate, and hyaluronic acid.

9. The growth factor delivery system in accordance with any one of claims 1-3, wherein the shell is treated with a cross-linking agent selected from the group consisting of ethyldimethylaminopropyl carbodiimide, genipin, and glutaraldehyde.

10. A method of preparing a core-shell structured, microcapsule type growth factor delivery system comprising:
preparing a polymeric microsphere comprising a first component that is necessary for differentiation or proliferation of cells; and
encapsulating said polymeric microsphere into another polymer comprising a second component that is necessary for differentiation or proliferation of cells to prepare a core-shell microcapsule, wherein at least one of the first component and the second component is a growth factor.

11. The method in accordance with claim 10, wherein both of the first and second components that are necessary for differentiation or proliferation of cells are growth factors, wherein each growth factor, independently, is at least one selected from the group of transforming growth factor (TGF-β), fibroblast growth factor (FGF), bone morphogenic protein (BMP), vascular endothelial growth factor (VEGF), epidermal growth factor (EGF), insulin-like growth factor (IGF), platelet-derived growth factor (PDGF), nerve growth factor (NGF), hepatocyte growth factor (HGF), epidermal growth factor, angiopoietin-1, angiopoietin-2, neurotrophin, placental growth factor (PIGF), granulocyte colony simulating factor (G-CSF), and granulocyte macrophage colony simulating factor (GM-CSF).

12. The method in accordance with claim 10, wherein either of the first or second component that is necessary for differentiation or proliferation of cells is a growth factor, and the other component is selected from the group consisting of heparin, animal growth hormone, human growth hormone, erythropoietin, interferon, follicle-simulating hormone, luteinizing hormone, goserelin acetate, leuprolein acetate, luteinizing hormone-releasing hormone agonist of decapeptyl, dexamethasone, ascorbate-2-phosphate, β-glycerophosphate, insulin, glucose, paclitaxel, rapamycin, and an anti-inflammatory agent.

13. The method in accordance with any one of claims 10 to 12, wherein the microspheres are made by using a method selected from the group consisting of a phase separation method, a spray-drying method, a solvent-evaporation drying method, and a low temperature solvent extraction method.

14. The method in accordance with any of claims 10 to 12, wherein the microsphere has interconnected multiple pores inside and a covered surface.

15. A method of preparing a core-shell structured, microcapsule type growth factor delivery system comprising:
encapsulating a polymer solution comprising a first component that is necessary for differentiation or proliferation of cells into another polymer comprising a second component that is necessary for differentiation or proliferation of cells to prepare a core-shell structured microcapsule, wherein at least one of the first component and the second component is a growth factor.

16. The method in accordance with claim 15, wherein both of the first and second components that are necessary for cell differentiation or proliferation are growth factors, wherein each growth factor, independently, is at least one selected from the group of transforming growth factor (TGF-β), fibroblast growth factor (FGF), bone morphogenic protein (BMP), vascular endothelial growth factor (VEGF), epidermal growth factor (EGF), insulin-like growth factor (IGF), platelet-derived growth factor (PDGF), nerve growth factor (NGF), hepatocyte growth factor (HGF), epidermal growth factor, angiopoietin-1, angiopoietin-2,neurotrophin, placental growth factor (PIGF), granulocyte colony simulating factor (G-CSF), and granulocyte macrophage colony simulating factor (GM-CSF).

17. The method in accordance with claim 15, wherein one of the first or second components that are necessary for differentiation or proliferation of cells is a growth factor, and the other component is selected from the group consisting of heparin, animal growth hormone, human growth hormone, erythropoietin, interferon, follicle-simulating hormone, luteinizing hormone, goserelin acetate, leuprolein acetate, luteinizing hormone-releasing hormone agonist of decapeptyl, dexamethasone, ascorbate-2-phosphate, β-glycerophosphate, insulin, glucose, paclitaxel, rapamycin, and an anti-inflammatory agent.

18. The method in accordance with any of claims 15 to 17, wherein the polymer solution comprising the first component is obtained by simultaneously dissolving the polymer and the first component in a solvent.

19. The method in accordance with any of claims 15 to 17, wherein the polymer solution comprising a first component is obtained by dissolving the polymer in a solvent and then suspending the first component in the resulting solution.

20. The method in accordance with any of claims 10 to 12 and claims 15 to 17, wherein preparation of the microcapsule is conducted by electrodropping.

21. The method in accordance with any of claims 10 to 12 and claims 15 to 17, further comprising:
coating the resulting microcapsules with a material selected from the group consisting of chitosan, protamine, gelatin, collagen, poly(ethyleneimine) (PEI), poly-L-lysine, dextran sulfate, and hyaluronic acid.

22. The method in accordance with any one of claims 10 to 12, and claims 15 to 17, further comprising:
enhancing the mechanical properties of the growth factor delivery system using a crosslinking agent selected from the group consisting of ethyldimethylaminopropyl carbodiimide, genipin, and glutaraldehyde.

23. The method in accordance with any one of claims 10 to 12, and claims 15 to 17, wherein either of the polymer comprising the first component or the polymer comprising the second component, or both are synthetic polymers selected from the group consisting of poly(glycolic acid) (PGA), poly(L-lactic acid) (PLLA), poly(lactic acid-co-glycolic acid) (PLGA), poly-ε-caprolactone (PCL), poly(L-lactic acid-co-caprolactone) (PLCL), poly(amino acid), polyanhydride, polyorthoester, polyethylene glycol, polyvinyl alcohol, biodegradable polyurethane, and copolymers thereof.

24. The method in accordance with any one of claims 10 to 12, and claims 15 to 17, wherein either of the polymer comprising the first component or the polymer comprising the second component, or both are natural polymers selected from the group consisting of collagen, alginate, gelatin, chitosan, fibrin, hyaluronic acid, hyaluronic acid derivatives, cellulose, cellulose derivatives, self-assembled peptide, and composites thereof.

25. The method in accordance with any one of claims 10 to 12, wherein a suspension of the polymer microspheres comprising the first component and the polymer solution comprising the second component are simultaneously released to prepare the microcapsules.

26. The method in accordance with any one of claims 15 to 17, wherein the polymer solution comprising the first component and the polymer solution comprising the second component are simultaneously released to prepare the microcapsules.

27. A stem cell differentiation method comprising bringing a growth factor delivery system in accordance with any one of claims 1 to 3 or a growth factor delivery system manufactured by a method in accordance with any one of claims 10 to 12 and claims 15 to 17 into contact with stem cells.

28. The method in accordance with claim 27, wherein the stem cells are selected from the group consisting of embryonic stem cells, bone marrow stem cells, adipose stem cells, umbilical cord blood stem cells, peripheral blood stem cells, hematopoietic stem cells, muscle stem cells, neural stem cells and induced pluripotent stem cells.

29. A proliferation and differentiation method of tissue cells comprising bringing a growth factor delivery system in accordance with any one of claims 1 to 3 or a growth factor delivery system manufactured by a method in accordance with any one of claims 10 to 12 and claims 15 to 17 into contact with tissue cells.

30. The method in accordance with claim 29, wherein the tissue cells are selected from the group consisting of myocardium cells, neural cells, chondrocytes, osteoblasts, osteoclasts, liver cells, pancreatic cells, endothelial cells, epidermal cells, smooth muscle cells, and intervertebral disc cells.

31. A composition for reconstruction or regeneration of tissues comprising a growth factor delivery system in accordance with any one of claims 1 to 3 or a growth factor delivery system manufactured by a method in accordance with any one of claims 10 to 12 and claims 15 to 17.
